(19) United States
(12) Patent Application Publication
(10) Pub. No.: US 2025/0387023 A1
(43) Pub. Date: Dec. 25, 2025

(54) EXTENDABLE CHIN AND FOREHEAD REST

(71) Applicant: NEW YORK UNIVERSITY, New York, NY (US)

(72) Inventors: Sarah Segal, New York, NY (US); Joel S. Schuman, New York, NY (US); Chaim Gadi Wollstein, New York, NY (US)

(73) Assignee: NEW YORK UNIVERSITY, New York, NY (US)

(21) Appl. No.: 19/112,150

(22) PCT Filed: Sep. 15, 2023
(86) PCT No.: PCT/US2023/074355

§ 371 Date: Mar. 14, 2025

Related U.S. Application Data
(60) Provisional application No. 63/407,475, filed on Sep. 16, 2022.

(51) Int. Cl. A61B 3/00

(52) U.S. Cl. CPC A61B 3/0083

(57) ABSTRACT

An ophthalmic imaging system includes a head support system configured to stabilize a patient's head. The head support system includes a chinrest. A chinrest body including a first base member coupled to a second base member by a chin adjuster defining a chin opening. The chin adjuster defines an axis along which the first base member and the second base member are movable relative to one another to adjust a diameter of the chin opening. A chinrest mounting assembly includes a chinrest support including a ball joint and a chinrest attachment defining an opening to receive the ball joint. The opening has a first grooved hole and a second grooved hole defining a first position and a second position for the chinrest. A forehead rest is configured to be coupled to the ophthalmic device and includes a flexible forehead support and a forehead adjuster.

### CROSS-REFERENCE TO RELATED APPLICATIONS

The present application claims the benefit of priority to U.S. Provisional Application No. 63/407,475, filed Sep. 16, 2022, the entire contents of which are incorporated herein by reference.

### GOVERNMENT RIGHTS

This invention was made with government support under Grant No. NEI-R01 EY013178, a grant from the National Institutes of Health and the National Eye Institute awarded to New York University School of Medicine. The government has certain rights in the invention.

### TECHNICAL FIELD

The present disclosure relates generally to an extendable chin and forehead rest.

### BACKGROUND

Ophthalmic imaging devices may include chin and forehead rests which lack adjustability from patient to patient. Specifically, chin and forehead rests cannot, in a stable and consistent manner, be adjusted according to each patient's specific forehead and chin dimensions. Lack of adjustability may cause discomfort for patients, resulting in patients moving during the imaging process. Movement increases the time required to complete the imaging process as well as increases the likelihood of artifacts. Artifacts can lead to issues in making an accurate diagnosis. For example, artifacts may cause the misdiagnosis of eye conditions, such as glaucoma. Or, an eye condition may not be diagnosed at an appropriate time for an efficient intervention to be enacted.

### SUMMARY

According to one embodiment, an ophthalmic imaging system includes a head support system configured to stabilize a patient's head. The head support system includes a chinrest configured to hold the patient's chin. The chinrest includes a chinrest body comprising a first base member coupled to a second base member by a chin adjuster and defining a chin opening, the chin adjuster defining an axis along which the first base member and the second base member are movable relative to one another to adjust a diameter of the chin opening. A chinrest mounting assembly is configured to mount the chinrest body to an ophthalmic device. The chinrest mounting assembly includes a chinrest support configured to support the chinrest body, the chinrest support including a ball joint. A chinrest attachment including a top portion defining an opening to receive the ball joint couples the chinrest support to the chinrest attachment. The opening has a first grooved hole and a second grooved hole defining a first position and a second position for the chinrest. The chinrest attachment defines a mount opening configured to encompass a portion of the ophthalmic device and couple the chinrest to the ophthalmic device. The head support system further includes a forehead rest. The forehead rest is configured to be coupled to the ophthalmic device comprising a flexible forehead support and a forehead adjuster. The forehead support has an adjustable width and an adjustable curvature.

According to another embodiment, an ophthalmic imaging system includes a head support system configured to stabilize a patient's head. The head support system includes a chinrest positioned to hold the patient's chin. The chinrest includes a chinrest body comprising a first base member coupled to a second base member by a chin adjuster and defining a chin opening. The chin adjuster defines an axis along which the first base member and the second base member are movable relative to one another to adjust a diameter of the chin opening. The chinrest also includes a chinrest mounting assembly configured to adjust a position of the chinrest bod. The chinrest mounting as assembly includes a chinrest support configured to support the chinrest body. The chinrest support includes a ball joint. A chinrest attachment includes a top portion defining an opening to receive the ball joint, coupling the chinrest support to the chinrest attachment, the opening having a first grooved hole and a second grooved hole defining a first position and a second position for the chinrest. The head support system further includes a forehead rest positioned to abut a patient's forehead, the forehead rest comprising a flexible forehead support and a forehead adjuster, the forehead support having an adjustable width and an adjustable curvature.

According to another embodiment, a head support system for stabilizing a patient's head during use of an ophthalmic device includes a chinrest. The chinrest includes a chinrest body. The chinrest body includes a first base member coupled to a second base member by a chin adjuster defining a chin opening. The chin adjuster defines an axis along which the first base member and the second base member are movable relative to one another to adjust a diameter of the chin opening. The chinrest includes a chinrest mounting assembly for mounting the chinrest body to the ophthalmic device. The chinrest mounting assembly includes a chinrest support configured to support the chinrest body, the chinrest support including a ball joint, and a chinrest attachment. The chinrest attachment includes a top portion defining an opening to receive the ball joint, coupling the chinrest support to the chinrest attachment, the opening having a first grooved hole and a second grooved hole defining a first position and a second position for the chinrest. The chinrest attachment also defines a mount opening configured to encompass a portion of the ophthalmic device and couple the chinrest to the ophthalmic device. The head support system also includes a forehead rest including a flexible forehead support and a forehead adjuster, the forehead support having an adjustable width and an adjustable curvature. The forehead rest also includes a forehead rest attachment for mounting the forehead rest to the ophthalmic device.

### BRIEF DESCRIPTION OF THE FIGURES

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

FIG. 1 is an image of a head support system coupled to an optical coherence tomography (OCT) system, according to one embodiment.

FIG. 2 is a perspective view of a chinrest assembly of the head support system of FIG. 1;

FIG. 3 is a perspective view a chinrest body of a chinrest assembly, according to one embodiment;

FIG. 4 is a top view of the chinrest body of FIG. 3;

FIG. 5 is an exploded view of a chinrest mount of a chinrest assembly, according to one embodiment;

FIG. 6 is a perspective view of a chinrest attachment of a chinrest mount, according to one embodiment;

FIG. 7 is a front view of the chinrest attachment of FIG. 6 coupled to an OCT system;

FIG. 8 is a perspective view of a chinrest assembly, according to another embodiment;

FIG. 9 is a perspective view of a chinrest mount of the chinrest assembly of FIG. 8;

FIG. 10 is a front perspective view of a forehead rest assembly of a head support system, according to one embodiment;

FIG. 11 is a perspective view of the forehead assembly of FIG. 10;

FIG. 12 is a front view of a forehead rest assembly, according to another embodiment;

FIG. 13 is a side view of the forehead rest assembly of FIG. 12;

FIG. 14 is a front view of a forehead rest assembly, according to another embodiment; and

FIG. 15 is a sectional view of the forehead rest assembly of FIG. 14.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Embodiments described herein relate generally to a head support system designed to comfortably stabilize a patient's head during an examination, such as an examination using an ophthalmic device. The head support system is designed to be easily sterilized between patients, safe, and durable. The head support system provides the patient with a comfortable resting position and properly stabilizes a patient's head. The head support system supports the weight of a patient's head and holds the head in a steady, fixed position. The head support system may be adjusted patient to patient to account for differences in chin and forehead dimensions. The head support system includes a chinrest in which a patient places their chin prior to an examination. The chinrest is adjustable to fit the dimensions of a patient's chin. The head support system further includes a forehead rest against which a patient rests their forehead. The forehead rest is adjustable to fit the dimensions and curvature of a patient's head.

The chinrest includes a chinrest body, and a chinrest mounting assembly. The chinrest body holds the patient's chin and includes a first base member second base member. The first base member and the second base member are coupled together by an chin adjuster, such as a set of rotary shafts. The first base member and the second base member define an opening and are configured to move away from one another via the adjuster and that holds the patient's chin. The chin adjuster allows a diameter of the opening to increase, adjusting to the dimensions of a patient's chin. The chinrest body may further include bearings, or other similar parts, to reduce friction between moving parts as the chinrest body is being adjusted. The chinrest body may include a locking mechanism to fix the position of the first base member and the second base member at a desired opening diameter. A cushion may be provided on the chinrest to improve patient comfort.

The chinrest mounting assembly couples the chinrest body to an device, such as an ophthalmic device. The device may be existing device or a new device. The chinrest mounting assembly is adjustable to the variable outer length of the chinrest body and is disposed under the chinrest body to provide support for the chinrest body. The chinrest mounting assembly is configured to extend and retract with the chinrest body. The chinrest mounting assembly includes a chinrest support having a ball joint, or other similar parts, to provide further rotational adjustment. Additional means of adjustment further improve the stability of the patient's head while being examined, or imaged, as well as provides a more comfortable patient experience. The chinrest mounting assembly further includes a chinrest attachment attaching the chinrest body to the device, using, for example, a clamp. The means of attachment may be customized to adapt to different devices.

The head support system further includes a forehead rest assembly including a forehead support. The forehead support may be formed of a single piece or multiple pieces. The forehead support adjusts to the size and curvature of a patient's forehead, and is configured to attach to a device.

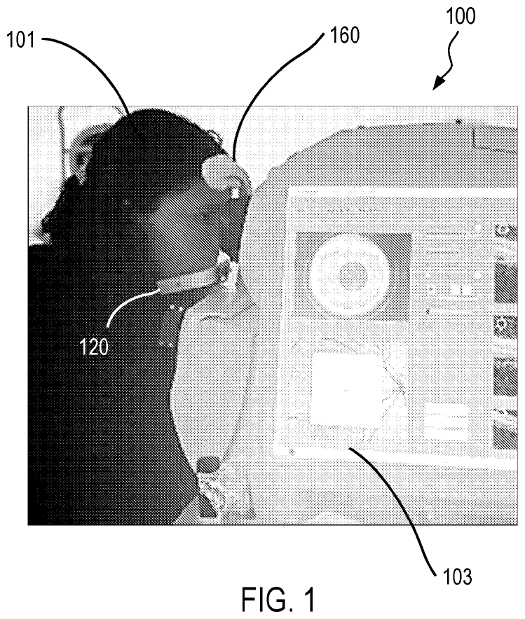

Referring to FIG. 1, the head support system 100 includes a chinrest 120 and a forehead rest 160. The head support system 100 is configured to be coupled to a device 103, such as an ophthalmic device (e.g., optical coherence tomography systems, slit lamps, auto refractometers). The head support system 100 may be adapted, or retrofitted, to existing devices or may be integrated into new devices. To position their head in the head support system 100, a patient 101 places their chin onto the chinrest 120 and places their forehead against the forehead rest 160.

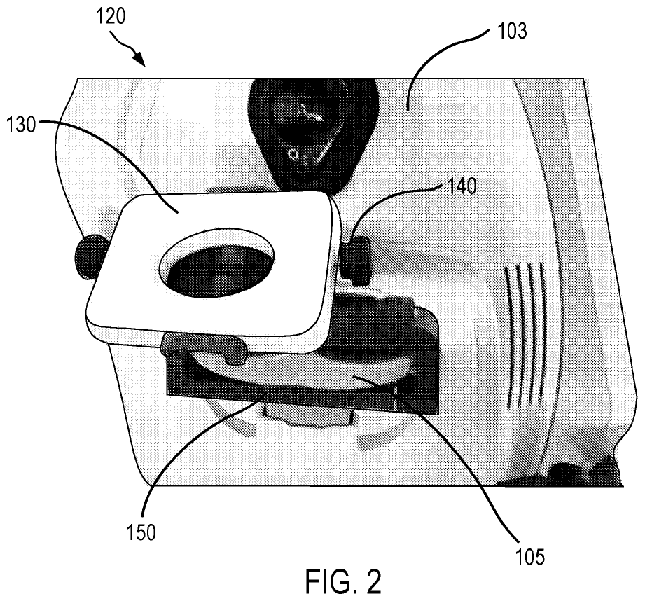

Referring to FIG. 2, the chinrest 120 includes chinrest body 130 having a chin adjuster 140 and a chinrest mounting assembly 150. The chinrest mounting assembly 150 may be used to attach the chinrest 120 to a device 103 having an existing chinrest 105. Unlike the existing chinrest 105, the chinrest 120 can be adjusted via the chin adjuster 140 to comfortably fit and position a range of chin sizes.

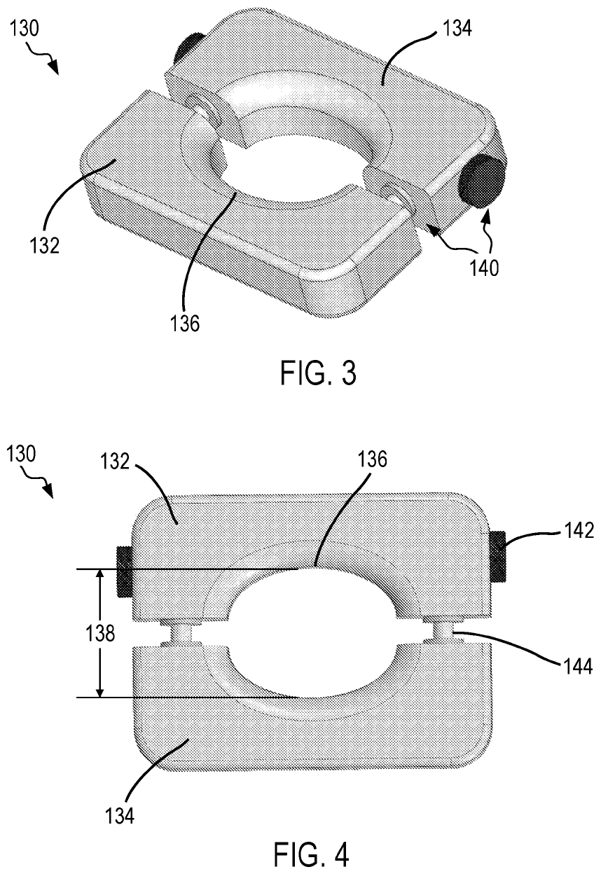

Referring to FIGS. 3-4, the chinrest body 130 includes a first base member 132, and second base member 134, and a chin adjuster 140. The first base member 132 and the second base member 134 define an opening 136 for a patient to position their chin. In some embodiments, the opening may be contoured to provide patient comfort. In some embodiments a silicone cover may be provided over the chinrest body 130 to provide additional patient comfort and ease of cleaning and/or sanitation. In some embodiments, the first base member 132 and the second base member 134 are identical. In some embodiments, the first base member 132 and the second base member 134 are different. The first base member 132 and the second base member 134 are coupled to one another by the chin adjuster 140. The chin adjuster 140 is configured to move the first base member 132 and the second base member 134 relative to one another such that an opening diameter 138 may increase or decrease based on the dimensions of a patient's chin. For example, moving the first base member 132 and the second base member 134 away from one another increases the opening diameter 138.

The chin adjuster 140 includes rotary shafts 144, including a rotary shaft on two opposing sides of the first base member and the second base member. The rotary shafts 144 extend into the first base member 132 and the second base member 134 and couple the first base member 132 and the second base member 134 together. The rotary shafts 144 define an axis along which the first base member 132 and the second base member 134 move relative to one another. In some embodiments, the rotary shafts 144 define a horizontal axis, and in some embodiments, the axis is not horizontal. The first base member 132 and the second base member 134 may also include bearings, or other means to reduce friction, configured move along the rotary shafts. Bearings allow for the first base member 132 and the second base member 134 to smoothly adjust along the rotary shafts 144, reducing wear of the chinrest 120.

The chin adjuster 140 further provides a position lock to fix the chinrest body 130 at a desired opening diameter 138. The position may be locked via thumb screws 142 positioned on opposite sides of the first base member 132. In some embodiments, the thumb screws 142 may be positioned on opposite sides of the second base member 134, allowing the second base member 134 to remain stationary or move along the rotary shafts 144. The thumb screws 142 are configured to rotate within the first base member 132 to allow the first base member 132 to remain stationary or move along the rotary shafts 144. For example, by tightening (e.g. rotating clockwise) the thumb screws 142, the first base member 132 may be locked in place on the rotary shafts 144 to hold a patient's head in a fixed position on the chinrest body 130. By loosening (e.g. rotating counterclockwise) the thumb screws 142, the first base member 132 may be moveable along the rotary shafts 144 so that the chinrest body 130 can be adjusted to fit varying chin dimensions, providing a comfortable position for patients and reducing patient movement. In some embodiments, the thumb screws 142 may be further configured to secure the first base member 132 to the rotary shafts 144. In some embodiments, the thumb screws 142 may include a textured (e.g. groove, roughed, knurled) surface to allow an operator to easily grip and adjust the thumb screws 142.

In some embodiments, the chin adjuster includes an electrically coupled remote controller configured to adjust the first base member 132 and/or the second base member 134 along the rotary shafts 144. In some embodiments, the chin adjuster 140 is motorized to provide an operator quicker, and more precise control. In some embodiments, the position of the chinrest body may be locked without the use of the thumb screws 142. The position may be locked using pins that click into preset locations.

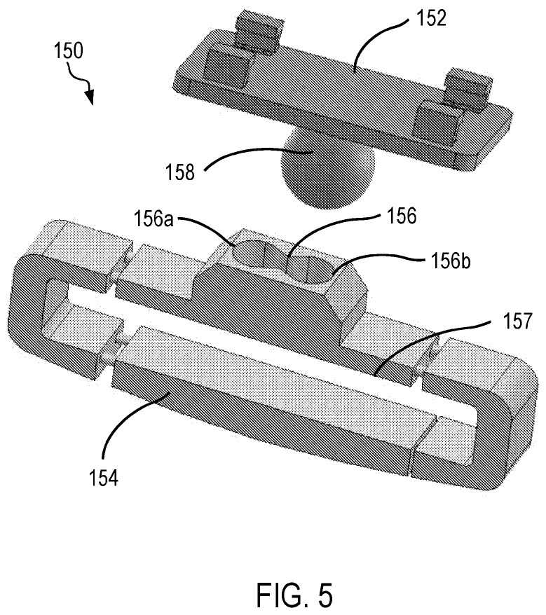

Referring to FIG. 5, the chinrest mounting assembly 150 is configured to couple the chinrest 120 to a device. The chinrest mounting assembly 150 includes a chinrest support 152 and a chinrest attachment 154. The chinrest support 152 is configured to couple to the chinrest body 130 and support the chinrest body 130. The chinrest support 152 includes a plate that the chinrest body 130 is placed on top of. The chinrest support 152 secures the chinrest body and couples to the chinrest attachment member via a ball joint 158. The ball joint 158 provides positional (e.g., rotational, angled, directional) adjustment of a patient's head. In some embodiments, the ball joint 158 allows rotation of the chinrest 120, and may include indents for spring plungers to set commonly used angles, or to set degree increments for adjustment. In some embodiments, the chinrest support 152 may include additional components, such as a hydraulic system, to allow for rotation of the chinrest, or to adjust the chinrest along an inclined plane.

The chinrest attachment 154 is configured to attach the chinrest 120 to a device. The chinrest attachment 154 defines a mount opening 157 to encompass a portion of a device and attach to the device. The chinrest attachment 154 includes a top portion defining an opening 156 having a first grooved hole 156a and second grooved hole 156b configured to receive the ball joint 158, securing the chinrest support 152 to the chinrest attachment 154. An operator may place the ball joint 158 in either the first grooved hole or the second grooved hole of the opening 156 to enable examination of the left or right eye of a patient. The chinrest attachment 154 may comprise of multiple sections coupled to one another (e.g., pins, rods, cylinders) to adjust the size of the mount opening 157, allowing for attachment to various existing devices. In some embodiments, the chinrest attachment 154 may be flexible and integrally formed. In some embodiments, the chinrest attachment 154 may include inserts on an inner surface of the opening to improve traction in attaching the chinrest 120 or prevent damage to the device. The inserts may be made out of silicone, or may be specifically tailored to fit existing devices.

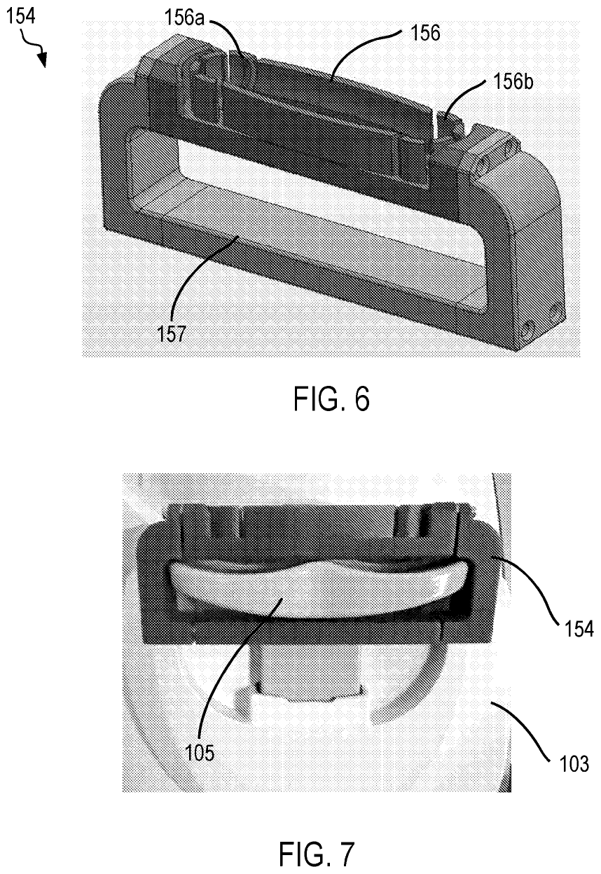

Referring to FIGS. 6-7, according to another embodiment, the chinrest attachment 154 is configured to attach to an existing chinrest 105 of a device 103. The chinrest attachment defines a mount opening 157 surrounding the existing chinrest 105 to securely attach the chinrest 120 to the device 103. In some embodiments, the mount opening 157 may be configured to attach to a portion of the existing chinrest 105, such as on a front/back side or left/right side. The chinrest attachment 154 includes a top portion defining an opening 156 including a first grooved hole 156a and a second grooved hole 156b configured to receive the ball joint 158, securing the chinrest support 152 to the chinrest attachment 154. An operator may place the ball joint 158 in either the first grooved hole or the second grooved hole of the opening 156 to move the chinrest 120 to a first position and a second position, enabling examination of the left or right eye of a patient.

In various embodiments, the chinrest mounting assembly 150 allows directional adjustment of the chinrest 120 to various positions to examine the left or right eye of a patient. In some embodiments, the chinrest attachment 154 does not include a first grooved hole 156a and a second grooved hole 156b. Instead, the chinrest attachment 154 may include a support member. The chinrest support 152 may couple to the support member and move, or slide, along a length of the support member to a various positions along the support member to examine the left or right eye of a patient. When the chinrest support 152 has been moved to a desired position along the support member, the chinrest support 152 may be locked into position. In some embodiments, the chinrest support 152 may be configured to be received in a slot or groove on the chinrest attachment 154. The chinrest support 152 may be secured within the slot and move along a length of the slot to various positions along the slot to examine the left or right eye of a patient.

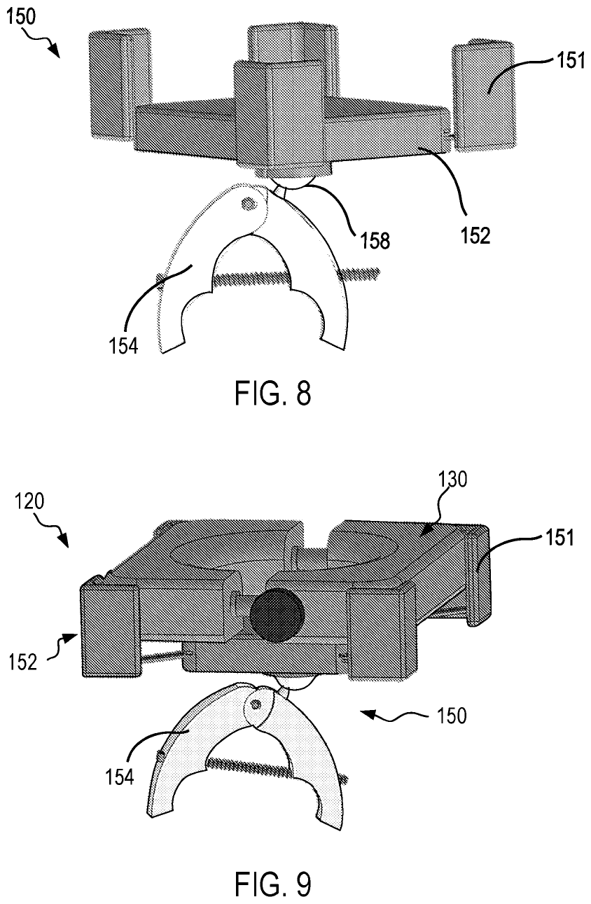

Referring to FIGS. 8-9, according to another embodiment, the chinrest mounting assembly 150 includes a chinrest support 152 and a chinrest attachment 154. The chinrest support 152 includes a plurality of grippers 151. The chinrest support 152 includes a plate to support for the chinrest body 130 and can be adjusted, by the plurality of grippers 151, to fit the variable length of the chinrest body 130. The plurality of grippers 151 is coupled to the corners of the chinrest support 152 and is configured to extend and retract with the chinrest body 130. The chinrest support 152 is then connected to the chinrest attachment 154 via a ball joint 406. The chinrest attachment 154 includes a clamp and threaded pin configured to secure the chinrest 120 to a device. The clamp may include two members coupled to one another that allow the chinrest attachment 154 to clamp around a range of devices, and a threaded rod to attach the chinrest attachment to a device.

In some embodiments, the chinrest support 152 may adapt to the size of the chinrest body 130 using a plurality of edge adjusters protruding outward from the chinrest support 152. Other embodiments may include four edge adjusters 404 positioned on each of the four corners of the chinrest support 152. The edge adjusters may include a plurality of springs biased towards the center of the chinrest support 152 to exert a compressive force on the outer edges of the chinrest body 130, securely coupling the chinrest body 130 to the chinrest support 152. In some embodiments, the chinrest attachment 154 may attach to devices using other attachment methods (e.g., fasteners, magnetic pins, pins and slots, grooved slots) to adapt to specific device models. For example, a custom chinrest attachment 154 may be developed to fit commonly found devices.

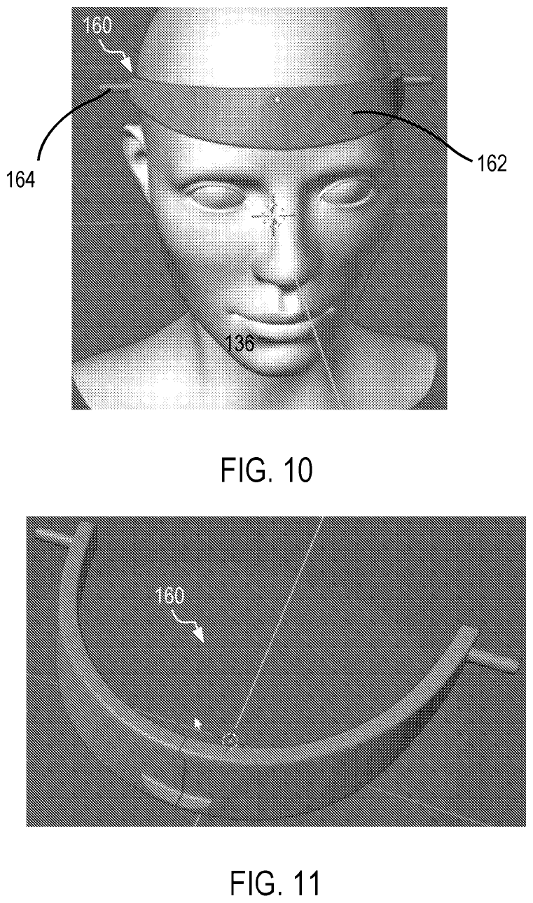

Referring to FIGS. 10-11, the forehead rest 160 is configured to position the front of a patient's head by providing a surface for the patient's forehead to rest against. The forehead rest 160 is configured to adjust to the width and curvature of a patient's head. The forehead rest 160 includes forehead support 162. In some embodiments, the forehead support 162 includes a male component and a female component. The female component is configured to receive the male component to create a flexible joint within the forehead support 162. The forehead rest 160 may also include pins 164, one protruding from each side of the forehead rest 160. The pins 164 are configured to be received in slots located on a device to adjust the patient's head relative to the device and mount the forehead rest 160 to the device. In some embodiments, the forehead rest 160 includes a quick release strap that may be tightened or loosened to adjust to the size of a patient's head. In some embodiments, the forehead support 162 may be adjusted via slotted grooves that can be locked using a set screw. In some embodiments, adjustment of the forehead support 162 is motorized to provide an operator quicker, and more precise control.

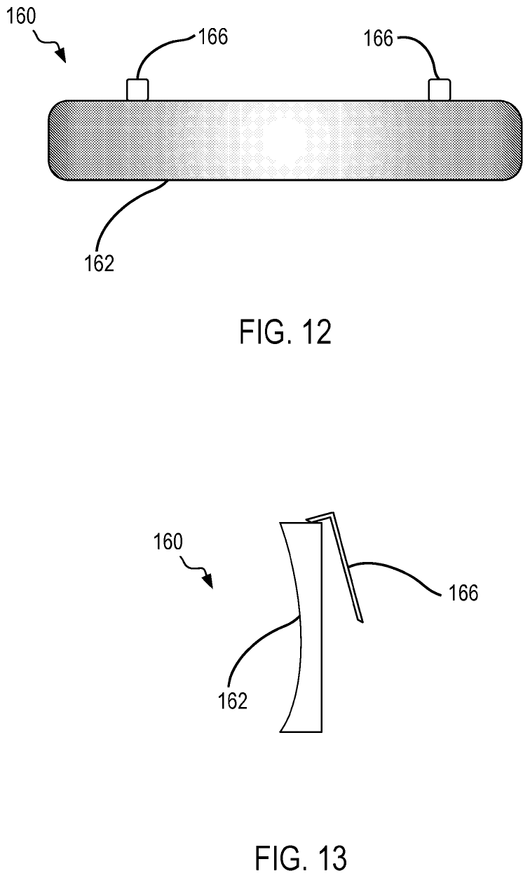

Referring to FIGS. 12-13, according to another embodiment, the forehead rest 160 includes a forehead support 162 and a plurality of clips 166. The forehead support 162 may be composed of ABS or another polymeric material. In some embodiments, the forehead support 162 includes a cushion to comfortably contact the patient's forehead. The cushion may be made of silicone, or similar materials that are easily cleaned or sanitized. The forehead support 162 is able to deform from its original shape to fit various patient forehead dimensions. Once removed from the patient's forehead, the forehead support 162 may revert back to its original shape. The forehead support 162 is coupled to a plurality of clips 166. In some embodiments, the plurality of clips 166 include two clips that are equally spaced from opposite ends of the forehead support 162. The plurality of clips 166 is configured to secure the forehead support 162 to a device, enabling the forehead rest 160 to be removeable and attachable to various devices. In some embodiments, an operator user may slide the plurality of clips 166 over a portion of a (e.g. a bar) to secure the forehead rest 160 to the device. Each clip in the plurality of clips 166 extends outward and downward from the forehead support 162 and may be composed of metal or plastic.

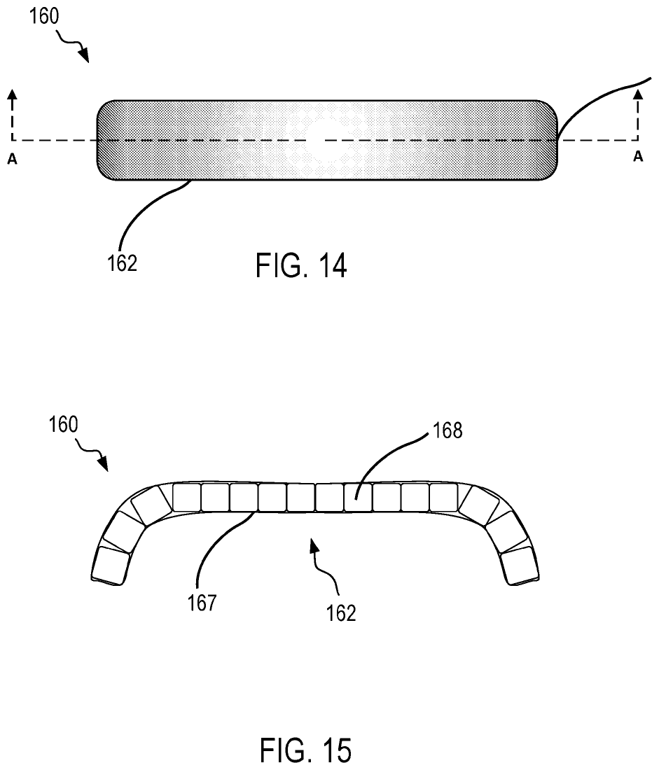

Referring to FIGS. 14-15, according to yet another embodiment, the forehead rest 160 includes a forehead support 162 that is a single piece having a flexible outer surface 167. Within the forehead support 162, a plurality of plates 168 are coupled to one another and arranged to allow the forehead support 162 to change the extension and curvature of the forehead rest 160. In some embodiments, the forehead rest 160 may be attached to a device using clamps, or a custom molded part allowing the forehead rest 160 to be snapped or pushed onto the device.

### Definitions

No claim element herein is to be construed under the provisions of 35 U.S.C. § 112 (f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any references herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Although the figures and description may illustrate a specific order of method steps, the order of such steps may differ from what is depicted and described, unless specified differently above. Also, two or more steps may be performed concurrently or with partial concurrence, unless specified differently above.

1. An ophthalmic imaging system, comprising:
a head support system configured to stabilize a patient's head, the head support system comprising:
a chinrest configured to hold the patient's chin, the chinrest comprising:
a chinrest body comprising a first base member coupled to a second base member by a chin adjuster and defining a chin opening, the chin adjuster defining an axis along which the first base member and the second base member are movable relative to one another to adjust a diameter of the chin opening; and
a chinrest mounting assembly configured to mount the chinrest body to an ophthalmic device, the chinrest mounting assembly comprising:
a chinrest support configured to support the chinrest body, the chinrest support including a ball joint; and
a chinrest attachment including a top portion defining an opening to receive the ball joint, coupling the chinrest support to the chinrest attachment, the opening having a first grooved hole and a second grooved hole defining a first position and a second position for the chinrest, the chinrest attachment further defining a mount opening configured to encompass a portion of the ophthalmic device and couple the chinrest to the ophthalmic device; and
a forehead rest configured to be coupled to the ophthalmic device comprising a flexible forehead support and a forehead adjuster, the forehead support having an adjustable width and an adjustable curvature.

2. The ophthalmic imaging system of claim 1, wherein the chin adjuster comprises:
a first rotary shaft extending between the first base member and the second base member on a first side of the chin opening; and
a second rotary shaft extending between the first base member and the second base member on a second side of the chin opening opposite the first side,
wherein the first base member and the second base member are movable along the first rotary shaft and the second rotary shaft.

3. The ophthalmic imaging system of claim 2, further comprising a first thumb screw and a second thumb screw securing the first base member or the second base member to the first rotary shaft and the second rotary shaft, the first thumb screw and the second thumb screw locking a position of the chinrest body at a desired diameter of the chin opening.

4. The ophthalmic imaging system of claim 1, further comprising a cushion coupled to the chinrest placed over the chin opening, the cushion forming a central opening configured to receive the patient's chin.

5. The ophthalmic imaging system of claim 1, wherein the chinrest support comprises a plurality of edge adjusters configured to extend the chinrest support in response to the first base member moving away from the second base member and retract the chinrest support in response to the first base member moving towards the second base member.

6. The ophthalmic imaging system of claim 1, wherein the forehead support comprises:
a female member; and
a male member, wherein the male member is configured to move linearly relative to the female member.

7. The ophthalmic imaging system of claim 1, wherein the forehead rest further comprises a plurality of pins extending from outer ends of the forehead support, the plurality of pins configured to fit into a slot on the device.

8. The ophthalmic imaging system of claim 1, wherein the forehead support comprises:
a forehead support body defining an outer surface; and
a plurality of plates linked to one another and disposed within the forehead support body, the plurality of plates being arranged along a width of the forehead support body, each plate in the plurality of plates being movable relative to one another.

9. An ophthalmic imaging system, comprising:
a head support system configured to stabilize a patient's head, the head support system comprising:
a chinrest positioned to hold the patient's chin, the chinrest comprising:
a chinrest body comprising a first base member coupled to a second base member by a chin adjuster and defining a chin opening, the chin adjuster defining an axis along which the first base member and the second base member are movable relative to one another to adjust a diameter of the chin opening; and
a chinrest mounting assembly configured to adjust a position of the chinrest body, the chinrest mounting as assembly comprising:
a chinrest support configured to support the chinrest body, the chinrest support including a ball joint; and
a chinrest attachment including a top portion defining an opening to receive the ball joint, coupling the chinrest support to the chinrest attachment, the opening having a first grooved hole and a second grooved hole defining a first position and a second position for the chinrest; and
a forehead rest positioned to abut a patient's forehead, the forehead rest comprising a flexible forehead support and a forehead adjuster, the forehead support having an adjustable width and an adjustable curvature.

10. The ophthalmic imaging system of claim 9, wherein the chin adjuster comprises:
a first rotary shaft extending between the first base member and the second base member on a first side of the chin opening; and
a second rotary shaft extending between the first base member and the second base member on a second side of the chin opening opposite the first side,
wherein the first base member and the second base member are movable along the first rotary shaft and the second rotary shaft.

11. The ophthalmic imaging system of claim 10, further comprising a first thumb screw and a second thumb screw securing the first base member or the second base member to the first rotary shaft and the second rotary shaft, the first thumb screw and the second thumb screw locking a position of the chinrest body at a desired diameter of the chin opening.

12. The ophthalmic imaging system of claim 9, further comprising a cushion coupled to the chinrest placed over the chin opening, the cushion forming a central opening configured to receive the patient's chin.

13. The ophthalmic imaging system of claim 9, wherein the chinrest support comprises a plurality of edge adjusters configured to extend the chinrest support in response to the first base member moving away from the second base member and retract the chinrest support in response to the first base member moving towards the second base member.

14. The ophthalmic imaging system of claim 9, wherein the forehead support comprises:
a female member; and
a male member, wherein the male member is configured to move linearly relative to the female member.

15. The ophthalmic imaging system of claim 1, wherein the forehead support comprises:
a forehead support body defining an outer surface; and
a plurality of plates linked to one another and disposed within the forehead support body, the plurality of plates being arranged along a width of the forehead support body, each plate in the plurality of plates being movable relative to one another.

16. A head support system for stabilizing a patient's head during use of an ophthalmic device comprising:
a chinrest comprising:
a chinrest body comprising a first base member coupled to a second base member by a chin adjuster and defining a chin opening, the chin adjuster defining an axis along which the first base member and the second base member are movable relative to one another to adjust a diameter of the chin opening; and
a chinrest mounting assembly for mounting the chinrest body to the ophthalmic device, the chinrest mounting assembly comprising:
a chinrest support configured to support the chinrest body, the chinrest support including a ball joint; and
a chinrest attachment including a top portion defining an opening to receive the ball joint, coupling the chinrest support to the chinrest attachment, the opening having a first grooved hole and a second grooved hole defining a first position and a second position for the chinrest, the chinrest attachment further defining a mount opening configured to encompass a portion of the ophthalmic device and couple the chinrest to the ophthalmic device; and
a forehead rest comprising:
a flexible forehead support and a forehead adjuster, the forehead support having an adjustable width and an adjustable curvature; and
a forehead rest attachment for mounting the forehead rest to the ophthalmic device.

17. The head support system of claim 16, wherein the chin adjuster comprises:
a first rotary shaft extending between the first base member and the second base member on a first side of the chin opening; and
a second rotary shaft extending between the first base member and the second base member on a second side of the chin opening opposite the first side,
wherein the first base member and the second base member are movable along the first rotary shaft and the second rotary shaft.

18. The head support system of claim 17, further comprising a first thumb screw and a second thumb screw securing the first base member or the second base member to the first rotary shaft and the second rotary shaft, the first thumb screw and the second thumb screw locking a position of the chinrest body at a desired diameter of the chin opening.

19. The head support system of claim 16, wherein the forehead support comprises:
a female member; and
a male member, wherein the male member is configured to move linearly relative to the female member.

20. The head support system of claim 16, wherein the forehead support comprises:
a forehead support body defining an outer surface; and
a plurality of plates linked to one another and disposed within the forehead support body, the plurality of plates being arranged along a width of the forehead support body, each plate in the plurality of plates being movable relative to one another.
